(19) United States
(12) Patent Application Publication
(10) Pub. No.: US 2015/0004106 A1
(43) Pub. Date: Jan. 1, 2015

(54) ACNE treatment method

(71) Applicant: Jordan Austin, Benson, AZ (US)

(72) Inventor: Jordan Austin, Benson, AZ (US)

(21) Appl. No.: 13/987,028

(22) Filed: Jun. 26, 2013

(51) Int. Cl. A61K 31/575

(52) U.S. Cl. CPC A61K 31/575
USPC 424/59; 514/182; 514/171

(57) ABSTRACT

A topical skin care method for acne and subcutaneous gland disorders utilizing a composition utilizing beta-sitosterol in conjunction with a dermatologically acceptable vehicle such as a lotion, gel, or cream. In some embodiments a sunscreen agent is also used to reduce skin exposure to ultraviolet rays. In yet another embodiment an antifungal medication additionally applied with the beta-sitosterol and dermatological vehicle to provide enhanced treatment of the acne and associated skin conditions.

### BACKGROUND OF THE INVENTION

This is a continuation in part of U.S. patent application Ser. No. 13/573,254, filed on Sep. 5, 2012, and entitled “Beta Sitosterol Acne Treatment”.

This invention relates generally to the topical treatment of skin conditions such as subcutaneous gland disorders and more particularly to the treatment of acne.

Acne (acne vulgaris) creates areas of skin with scaly red skin (seborrhea), papules, comedones, pustules, nodules, and scaring. While the affects usually dissipate or diminish by the age of twenty-five, during adolescence, acne can also cause severe psychological problems as severe acne provides an image problem for teens already struggling with learning social skills.

In extreme cases, acne can cause permanent scarring. Acne appears mostly on the face but can also appear on the chest and back.

The cause of acne is generally considered caused by a change in the testosterone level (both in males and females) but is exasperated by hormonal activity (females predominately), genetic predisposition, psychological stress, infections, and even diet.

The traditional treatment uses benzoyl peroxide with more pronounce cases requiring antiseptics, antibiotics, hormone replacement, topical retinoids, oral retinoids, anti-inflammatories, nicotinamide, Ibuprofen with tetracycline, and even mandelic acid. In extreme cases, dermabrasion and phototherapy has also been attempted.

Many of these techniques have undesired side-affects, are difficult, or are expensive.

It is clear there is a need for an effective treatment of acne.

### SUMMARY OF THE INVENTION

A topical skin care composition for acne which utilizes beta-sitosterol in conjunction with a dermatologically acceptable vehicle in a lotion, gel, or cream form.

Beta-Sitosterol is commercially obtained through a variety of sources via the Internet from such sites as alibaba.com, urinozinc.com, and puritan.com. Techniques for obtaining Beta-Sitosterol are well known in the art and include, but are not limited to those described in: U.S. Pat. No. 4,265,824, entitled “Process for the Isolation of Beta-Sitosterol containing a Low percentage of alpha-Sitosterol; U.S. Pat. No. 4,298,539, entitled “Process for the Isolation of Beta-Sitosterol” issued to Koskenniska on Nov. 3, 1981; and U.S. Pat. No. 4,422,974, entitled “Process for the Purification of Beta-Sitosterol Isolated from the Unsaponifiables in Crude Soap from the Sulphate Cellulose Process” issued to Hamunen on Dec. 27, 1983; and, U.S. Pat. No. 4,153,622, entitled “Process for the Recvder of Beta-Sitosterol” issued to Lamminkari et al. on May 8, 1979; all of which are incorporated hereinto by reference.

Although the concentration of Beta-Sitosterol to dermatological vehicle is chosen to be an effective amount, it has been found that a concentration of Beta-Sitosterol of between two and nine percent by volume is ideal.

Lotion is the preferred dermatological vehicle. Lotions have a low to medium viscosity and are often used in faced creams. The lotion consistency preferred for this invention is an oil-in-water emulsion with cetearyl alcohol to maintain the emulsion.

Gels are more solid and are used by this invention for the application of the beta-sitosterol in a more concentrated form on the treatment site.

Other topical skin care formulations and dermatologically acceptable vehicles are well known to those of ordinary skill in the art and include, but are not limited to those described in: U.S. Pat. No. 8,173,184, entitled “Topical Skin Care Formulations Comprising Botanical Extracts” issued to Florence et al. on May 8, 2012; and U.S. Pat. No. 8,178,106, entitled “Topical Skin Care Formulations” issued to Hines et al. on May 15, 2012; and, United States Publication US 2002/0155180, entitled “Topical Preparation for Treating Acne and Hirsutism” issued to Goodman on Oct. 24, 2002; all of which are incorporated hereinto by reference.

The use of Beta-Sitosterol is well known to those of ordinary skill in the art for internal application and include, but is not limited to: U.S. Pat. No. 5,747,464, entitled Dietary Supplement Incorporating Beta-Sitosterol and Pectin” issued to See on May 5, 1998; U.S. Pat. No. 5,118,671, entitled “Complexes of Aescin, Beta-Sitosterol or Cholesterol, and Phospholipids and Pharmaceutical Compositions Containing Them” issued to Bombardelli et al. on Jun. 2, 1992; U.S. Pat. No. 8,168,234, entitled “Compositions that Treat or Inhibit Pathological Conditions Associated with Inflammatory Response” issued to Tripp et al. on May 1, 2012; U.S. Pat. No. 8,142,819, entitled “Synergistic Compositions that Treat or Inhibit Pathological Conditions Associated with Inflammatory Response” issued to Tripp et al. on Mar. 27, 2012; all of which are incorporated hereinto by reference.

In some embodiments, a sunscreen agent is also used with the Beta-Sitosterol/vehicle combination to reduce skin exposure to ultraviolet rays. Such sunscreen agents are organic or inorganic in nature and are applied as lotions, sprays, or gels. In one embodiment of the invention, the beta-sitosterol is contained within a spray applicator which contains the sunscreen as well. Such spray applicators are available in pump or aerosol modes and are ideal for application over a larger are of skin.

Aerosol sprays are dispensing systems which create an aerosol mist of liquid particles from a can under pressure. Once the mixture is released under pressure, the beta-sitosterol and dermatologically acceptable vehicle are propelled against the skin site for application.

In one embodiment of the invention for the treatment for skin conditions, an anti-fungal medication is combined with the beta-sitosterol and carrier. Such treatment is ideal for the treatment of Dermatophytosis (ringworm). In this manner, the medication not only treats the acne itself but also any secondary fungal infections which often accompany acne.

The ideal anti-fungal is Coricosteroid which includes a steroid hormone which has been shown to regulate inflammation.

Those of ordinary skill in the art readily recognize a variety of antifungal medications, including, but not limited to, Polyene antifungals, Imidazole, triazole, and thiazole antifunagals.

Another advantage of the use of beta-sitosterol by the present invention is beta-sitosterol's attribute as an anti-DHT (Dihydrotestosterone). Beta-sitosterol reduces seburm production in over active sebaceous glands, thereby inhibiting DHT. DHT has the characteristic of enhancing hair follicles. In the treatment of acne, the beta-sitosterol, as an anti-DHT, helps to reduce the number of follicles which contribute to the acne problem; in women, the anti-DHT characteristic assists in removing unwanted facial hairs.

Another advantage realized through the use of beta-sitosterol is that it reduces the number of clogged pores and also reduces pore size itself. This reduces uneven skin tone, wrinkles, and age spots. This also significantly reduces the formation of subcutaneous gland disorders.

In some applications, a quantity of beta-sitosterol is also mixed with makeup, toner, and face wash to expand the ease of use.

The invention, together with various embodiments of the invention will be more fully explained by the accompanying drawings and the following descriptions thereof.

### DRAWINGS IN BRIEF

FIG. 1 is the chemical structure for Beta-Sitosterol.

FIG. 2 graphically illustrates the combining of beta-sitosterol with a dermatological vehicle.

FIG. 3 illustrates an embodiment of the invention utilizing an aerosol applicator.

FIG. 4 is a table indicating the results found in a case study.

### DRAWINGS IN DETAIL

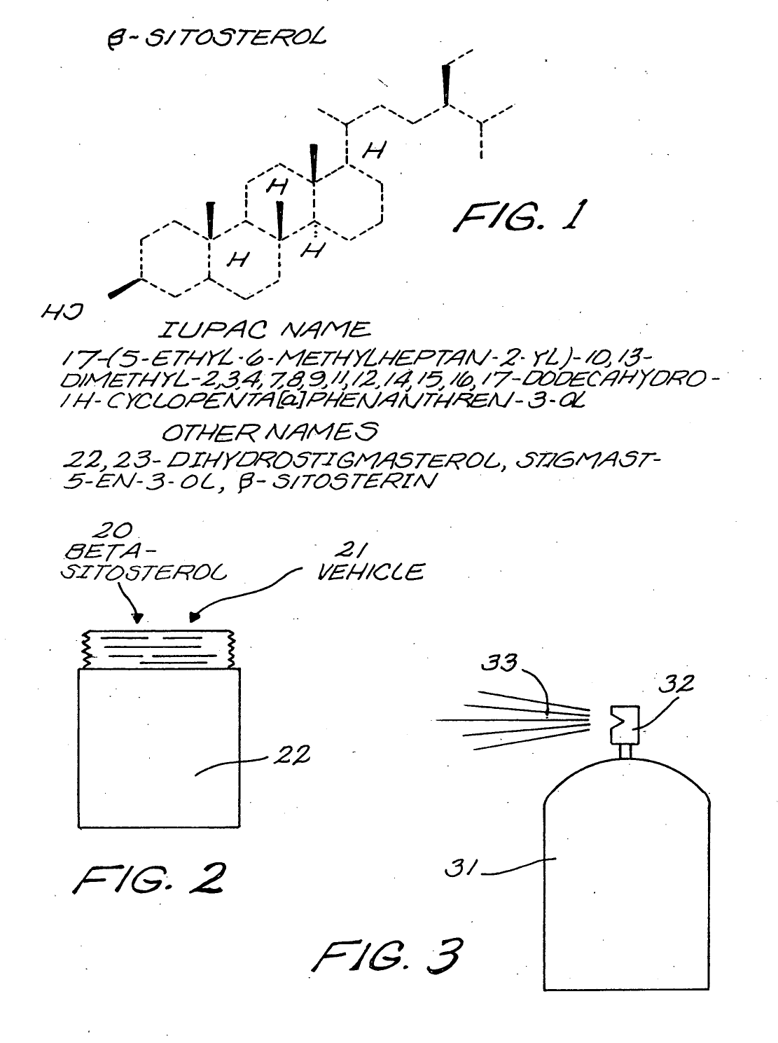

FIG. 1 is the chemical structure for Beta-Sitosterol.

Beta-Sitosterol is structurally similar to cholesterol and is typically derived from plant sterols. Beta-Sitosterol is hydrophobic and soluble in alcohols. The IUPAC name for Beta-Sitosterol is:
17-(5-Ethyl-6-methylheptan-2-yl)-10-13-dimethyl-2,3,4,7,8,9,11,12,14,16,17-deodeccahydrom-1H-cyclopental(alpha)phenanthren-3-ol

Found in a variety of plants. Beta-Sitosterol is found in such plants as: pecans, avocados, pumpkin seed, cashew fruit, rice bran, wheat germ, corn oils, soybeans and variety of other plants.

FIG. 2 graphically illustrates the combining of beta-sitosterol with a dermatological vehicle.

As illustrated, beta-sitosterol 20 is combined with a dermatological vehicle 21 and placed in a receptacle 22 for use for the treatment of acne. The combination is then applied by the user either on spot applications or a covering of the entire site where acne is occurring.

Although the concentration of beta-sitosterol 20 to vehicle 21 is selected to be effective against acne, it has been found that a concentration of between two and nine percent by weight has proven to be effective.

The ideal vehicle has been found to use as its core ingredients 3% beta-sitosterol, Niacinamide (vitamin B-3), Zinc Oxide, and Allantoin.

This vehicle containing the beta-sitosterol is applied to the sites of the subcutaneous gland disorders on a twice daily basis (morning and evening).

FIG. 3 illustrates an embodiment of the invention utilizing an aerosol applicator.

This embodiment of the invention is ideal for applying beta-sitosterol in conjunction with either an antifungal medication or a sunscreen. The combination of beta-sitosterol, dermatological vehicle, and secondary treatment (antifungal or sunscreen) is contained within vessel 31. Vessel 31, in this illustration, is pressurized; but, the invention is not intended to be limited to only pressure vessels but is also applicable to ambient containers which utilize a manually activated pump mechanism to dispense the combination as a spray.

When valve 32 is manually depressed, a spray of beta-sitosterol, dermatological vehicle, and secondary treatment (antifungal or sunscreen) 33 is ejected and is easily applied to the desired site. In some situations, the lotion is applied across the entire area which is prone to acne (such as the face) to act as a prophylactic against acne and other subcutaneous gland disorders.

When an antifungal is used with the beta-sitosterol, a more complete skin treatment is obtained. When a sunscreen is used with the beta-sitosterol, the skin is protected from ultra-violet rays and treated for acne at the same time.

As with the lotion embodiment described in FIG. 2, the application of the combination is sprayed onto the affected sites ideally twice a day.

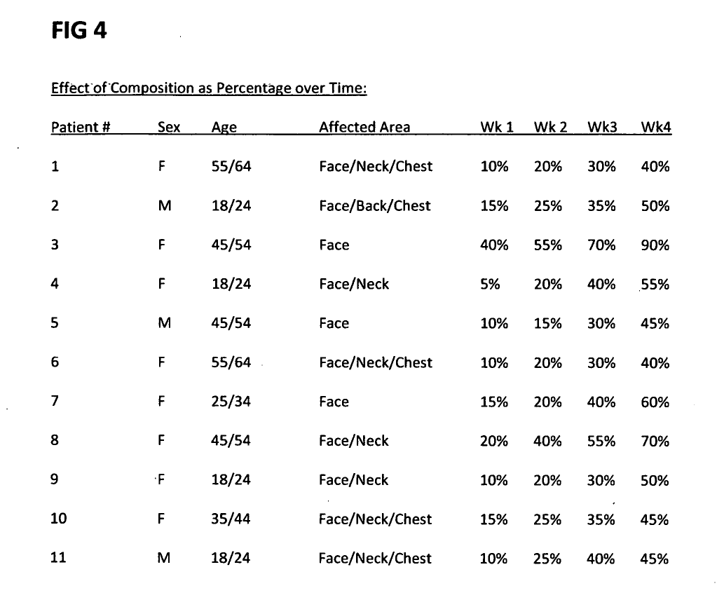

FIG. 4 is a table indicating the results found in a case study.

The study used a cross section of the population having a distribution of sexes, ages, and areas being affected. With all cases, there was an immediate reduction in the acne in the affected areas. With continuous use, in all of the subjects, further reductions in acne were achieved.

The study indicates that the first week's mean results for women show a 10% reduction in acne and with men the same result, 10%. At the end of the study, after four weeks of application, the women found a fifty percent reduction with the men showing a forty-five percent reduction.

This study confirms the efficacy of the treatment.

It is clear that the present invention provides for an effective treatment of acne.

What is claimed is:

1. A method for the treatment of acne comprising the step of regularly applying a composition having an effective amount of beta-sitosterol in a dermatologically acceptable vehicle to a site of acne.

2. The method for the treatment of acne according to claim 1, further including the step of applying, as the dermatologically acceptable vehicle, a lotion to soften the site of acne.

3. The method for the treatment of acne according to claim 2, further including the step of, simultaneous with the application of the composition, applying a sunscreen agent to the site of acne.

4. The method for the treatment of acne according to claim 1, further including the step of, simultaneous with the application of the composition, applying an effective amount of an antifungal medication to the site of acne.

5. The method for the treatment of acne according to claim 1, further including the step of creating an aerosol of said beta-sitosterol in a dermatologically acceptable vehicle at the time of application.

6. The method for the treatment of acne according to claim 1, further including the step of manually applying the beta-sitosterol to the site of acne.

7. A method of skin care comprising the step of, simultaneously,
a) applying an effective amount of beta-sitosterol for the treatment of a subcutaneous gland disorder; and,
b) applying a sunscreen agent adapted to reduce skin exposure to ultraviolet rays; and,
c) applying a dermatologically acceptable vehicle.

8. The method of skin care according to claim 7, further including the step of spraying the beta-sitosterol, the sunscreen agent, and the dermatologically acceptable vehicle onto the skin.

9. The method of skin care according to claim 7, further including the step of manually applying the beta-sitosterol, the sunscreen agent, and the dermatologically acceptable vehicle to the skin.

10. A method of skin care comprising the step of, simultaneously,
a) applying an effective amount of beta-sitosterol for the treatment of a skin disorder; and,
b) applying an effective amount of an antifungal medication; and,
c) applying a dermatologically acceptable vehicle.

11. The method of skin care according to claim 10, further including the step of applying a lotion for softening of the skin simultaneously with the application of the beta-stosterol.
